(19) United States
(12) Patent Application Publication
(10) Pub. No.: US 2026/0232748 A1
(43) Pub. Date: Aug. 13, 2026

(54) COMPOSITION FOR PREVENTING HAIR LOSS OR PROMOTING HAIR GROWTH COMPRISING CHIA SEED MUCILAGE

(71) Applicant: INDUSTRY-ACADEMIC COOPERATION FOUNDATION, YONSEI UNIVERSITY, Seoul (KR)

(72) Inventors: Yong SHIN, Seoul (KR); Zhen QIAO, Seoul (KR)

(21) Appl. No.: 19/533,601

(22) Filed: Feb. 9, 2026

(30) Foreign Application Priority Data
Feb. 12, 2025 (KR) 10-2025-0018022

(51) Int. Cl. A61K 36/537; A61K 47/10; A61K 47/32; A61P 17/14

(52) U.S. Cl. CPC A61K 36/537; A61K 47/10; A61K 47/32; A61P 17/14; A61K 2236/331; A61K 2236/53

(57) ABSTRACT

An embodiment relates to a composition for preventing hair loss or promoting hair growth, comprising chia seed mucilage. It was confirmed that a chia seed mucilage polysaccharide gel and a chia seed-based microencapsulation cream comprising chia seed mucilage according to the present invention exhibit antibacterial effects against bacteria and fungi, and regulate the hair growth cycle by inhibiting apoptosis and enhancing metabolic substances, thereby promoting hair follicle neogenesis and hair growth. In addition, since the composition is a natural plant-derived material with no toxicity and can be produced at low cost, and has been confirmed to be a material having superior effects compared to existing hair loss treatments, it can be utilized for preventing and treating hair loss.

### CROSS-REFERENCE TO RELATED APPLICATIONS

This application claims priority from Korean Patent Application No. 10-2025-0018022, filed on February 12, 2025, in the Korean Intellectual Property Office, which is incorporated herein by reference in its entirety.

### BACKGROUND OF THE INVENTION

### Field of the Invention

The present invention relates to a composition for preventing hair loss or promoting hair growth, comprising chia seed mucilage.

### Description of the Related Art

Hair is divided into a hair root and a hair shaft, and hair grows from a hair follicle (HF) surrounding the hair root. Two important factors that regulate biological characteristics in the hair follicle are outer root sheath (ORS) cells, which are hair follicle epithelial cells, and dermal papilla (DP) cells derived from mesenchymal tissue, and hair grows and falls out through repetition of the hair cycle. The hair growth cycle consists of three stages: anagen phase, apoptosis-driven catagen phase, and telogen phase (L. Bai, Y. Wang, K. Wang, X. Chen, Y. Zhao, C. Liu, X. Qu, Advanced Materials 2024, 36, 2311459.). The duration of each stage varies depending on individual age, hormonal factors, nutrition and health status, and genetic predisposition. An increase in the number and size of hair follicles is observed during the anagen phase, and unlike hair follicles in the telogen phase, which are located in the dermis, hair follicles in the anagen phase are located in deep subcutaneous tissue. Accordingly, increases in the number and size of hair follicles are closely associated with the anagen phase. By extending the duration of the anagen phase, which is the main stage of hair growth, the activation time of hair follicles is increased, thereby maintaining more hair follicles in a growth state simultaneously (N. Natarelli, N. Gahoonia, R. K. Sivamani, Journal of Clinical Medicine 2023, 12, 893.), and overall hair volume and density can be improved (Y. Shimizu, E. H. Ntege, H. Sunami, Y. Inoue, Regen Ther 2022, 21, 527.).

In animals including humans, hair follicles undergo multiple cycles of degeneration and regeneration throughout life beginning from the fetal developmental stage. In human hair growth, hair follicles continue to enlarge from the neonatal period through adolescence, resulting in progressively thicker hair. Hair follicles are composed of various types of cells, such as epithelial cells and mesenchymal cells. Mesenchymal cells serve as inductive organizers of hair follicles during fetal development and after birth. Follicle germ cells, which are bulbar matrix cells, are responsible for most mitotic proliferation within the hair follicle. During development, cells within the bulbar matrix migrate upward and proliferate, differentiating into hair matrix cells and inner and outer root sheath cells. The hair matrix group located at the central axis of the hair follicle continues to differentiate into cells forming the medulla, hair cortex, and hair cuticle.

Since hair plays an important role in improving external appearance, the importance of research on hair growth, hair enrichment, and prevention of hair loss has recently been increasing. In particular, as lifestyles have become westernized, quality of life has improved while stress has increased, and concerns regarding hair loss and scalp diseases have expanded to become matters of interest for all people. Hair loss or alopecia is a condition in which hair is lost. In hair loss, hair loss is not limited to scalp hair and may occur in any part of the body. Various types of alopecia are known, for example, alopecia areata, androgenetic alopecia, postmenopausal alopecia, female pattern alopecia, seborrheic alopecia, alopecia pityroides, senile alopecia, chemotherapy-induced alopecia, radiation-induced alopecia, alopecia caused by trichotillomania, and postpartum alopecia. Hair growth-promoting treatment methods known to date have been reported to have limited efficacy or adverse side effects. Representative hair loss treatments approved by the U.S. Food and Drug Administration (FDA) include topical minoxidil and oral Propecia, which are synthetic pharmaceuticals, and various side effects thereof have continuously been reported. Propecia exerts its effect by inhibiting conversion of testosterone into dihydrotestosterone (DHT), which is a causative substance of hair loss, but has been reported to potentially reduce male libido. Minoxidil was originally developed as an antihypertensive agent and later developed as a hair growth agent after reports that it promotes hair growth through activation of hair follicles, but various side effects such as skin disorders have been reported.

### SUMMARY OF THE INVENTION

An object of the present invention is to provide a pharmaceutical composition for preventing or treating hair loss.

Another object of the present invention is to provide a cosmetic composition for preventing hair loss or promoting hair growth.

Another object of the present invention is to provide a method for preparing a chia seed mucilage polysaccharide (chia seed mucilage polysaccharides, CSMP) gel.

Another object of the present invention is to provide a method for preparing a chia seed-based microencapsulation (Chia Seed-based Microencapsulation, CSMi) cream.

In order to solve the above-described problems, the present invention provides a pharmaceutical composition for preventing or treating hair loss, comprising chia seed mucilage.

In addition, the present invention provides a cosmetic composition for preventing hair loss or promoting hair growth, comprising chia seed mucilage.

In addition, the present invention provides a method for preparing a chia seed mucilage polysaccharide gel.

Further, the present invention provides a method for preparing a chia seed-based microencapsulation cream.

The chia seed mucilage polysaccharide gel and the chia seed-based microencapsulation cream comprising chia seed mucilage according to the present invention exhibit antibacterial effects against bacteria and fungi, and it has been confirmed that they regulate the hair growth cycle by inhibiting apoptosis and enhancing metabolic substances, thereby promoting hair follicle neogenesis and hair growth. In addition, since they are natural plant-derived materials with no toxicity and can be produced at low cost, and have been confirmed to exhibit superior effects compared to existing hair loss treatments, they can be utilized for preventing and treating hair loss.

### BRIEF DESCRIPTION OF THE DRAWINGS

FIGS. 1A to 1H are a diagram illustrating a preparation process of a chia seed-based microencapsulation (Chia Seed-based Microencapsulation, CSMi) cream and analysis of characteristics thereof:

A: schematic diagram of synthesis of the CSMi cream;

B: SEM images of the CSMi cream;

C: particle size of the CSMi cream;

D: results of viscosity analysis of a CSMP gel and the CSMi cream;

E: results of FTIR analysis of CSO, the CSMP gel, and the CSMi cream;

F: results of LC/MS analysis of the CSMP gel and the CSMi cream;

G: results of confocal microscopy analysis of the CSMi cream; and

H: schematic diagram of a microcapsule structure of the CSMi cream.

FIGS. 2A to 2F are a diagram illustrating analysis of cellular biological characteristics of CSO (Chia Seed Oil), the CSMP gel, and the CSMi cream:

A: results of cytotoxicity analysis;

B and C: results of cell migration analysis;

D and E: results of antibacterial effect analysis; and

F: results of western blot analysis.

FIGS. 3A to 3F are a diagram illustrating analysis of in vivo hair growth effects of CSO, the CSMP gel, and the CSMi cream:

A: in vivo experimental schedule;

B: hair growth images;

C: quantitative analysis results of hair coverage;

D: H&E staining images of dorsal skin sections;

E: quantification results of hair follicle neogenesis on day 21; and

F: histological analysis images of mice on day 21.

FIGS. 4A to 4E are a diagram illustrating analysis of effects of the CSMi cream on regulation of hair follicle heterogeneity:

A: schematic diagram of a single-cell analysis process;

B: UMAP plots of all cells derived from an untreated group and a CSMi cream-treated group;

C: dot plots showing candidate marker genes specific to each cell type;

D: bar plots showing proportions of cells differing between the untreated group and the CSMi cream-treated group;

E: schematic diagram summarizing spatial locations of hair growth-related subpopulations;

FIGS. 5A to 5D are a diagram illustrating analysis of effects of the CSMi cream on regulation of inner and outer layer heterogeneity of hair follicles:

A: UMAP plots of inner layer hair follicle cells in the untreated group and the CSMi cream-treated group;

B: UMAP plots of outer layer hair follicle cells in the untreated group and the CSMi cream-treated group;

C: bar plots showing proportions of cell clusters in the untreated group and the CSMi cream-treated group; and

D: results of pseudotime trajectory analysis of cells in the outer layer of hair follicles.

FIGS. 6A to 6E are a diagram illustrating analysis of effects of the CSMi cream on induction of glycolysis and autophagy:

A: results of KEGG pathway enrichment analysis of gene expression data;

B: GSEA levels of the glycolysis/gluconeogenesis pathway;

C: heatmap showing glycolysis-related pathways enriched in skin of mice in the untreated group and the CSMi cream-treated group;

D: DEG volcano plots related to autophagy in representative hair follicle cell types; and

E: heatmap showing differences in strength of intercellular interactions in the CSMi cream-treated group.

FIGS. 7A to 7C are a diagram illustrating effects of the CSMi cream on inhibition of apoptosis in hair follicle layers:

A: volcano plots of DEGs related to apoptosis in all cell types;

B: Mcl1 expression visualized on UMAP; and

C: Aven expression visualized on UMAP.

FIG. 8 is a diagram illustrating a mechanism of hair growth promotion by the CSMi cream.

### DETAILED DESCRIPTION OF THE INVENTION

Hereinafter, the present invention will be described in detail with reference to the accompanying drawings by way of exemplary embodiments of the present invention. However, the following embodiments are presented merely as examples of the present invention, and detailed descriptions of techniques or configurations that are well known to those skilled in the art may be omitted if it is determined that such descriptions unnecessarily obscure the gist of the present invention, and the present invention is not limited thereby. The present invention is capable of various modifications and applications within the scope of the claims set forth below and equivalents thereof.

In addition, the terminology used herein is employed to appropriately describe preferred embodiments of the present invention, and may vary depending on the intention of a user or operator or the custom in the relevant field to which the present invention pertains. Accordingly, definitions of such terms should be understood based on the overall contents of this specification. Throughout the specification, when any part is described as “comprising” a certain component, it means that other components may be further included rather than excluded, unless otherwise specifically stated.

Throughout the specification, “%” used to indicate a concentration of a specific substance refers to (w/w)% for solid/solid, (w/v)% for solid/liquid, and (v/v)% for liquid/liquid, unless otherwise specified.

In one aspect, the present invention relates to a pharmaceutical composition for preventing or treating hair loss, comprising chia (Salvia hispanica) seed mucilage.

In one embodiment, the chia seed mucilage may be chia seed mucilage prepared by a method comprising mixing chia seeds with distilled water and stirring; collecting mucilage; and filtering.

In one embodiment, the composition may comprise a gel comprising chia seed mucilage.

In one embodiment, the gel comprising chia seed mucilage may be a chia seed mucilage polysaccharide (chia seed mucilage polysaccharides, CSMP) gel prepared by a method comprising mixing and stirring chia seed mucilage with PVA (poly(vinyl alcohol)); adding and stirring D-sorbitol and glycerin; and performing UV treatment.

In one embodiment, the composition may further comprise chia seed oil (chia seed oil, CSO).

In one embodiment, the composition may comprise a gel comprising chia seed mucilage and a microencapsulation cream comprising chia seed oil.

In one embodiment, the microencapsulation cream may be a chia seed-based microencapsulation (Chia Seed-based Microencapsulation, CSMi) cream prepared by a method comprising mixing and stirring SDS (sodium dodecyl sulfate) with chia seed oil; adding and stirring a gel comprising chia seed mucilage; and performing UV treatment.

In one embodiment, microcapsules of the microencapsulation cream may have a particle size of 500 to 5000 nm.

In one embodiment, the composition may have antibacterial activity, anti-inflammatory activity, promotion of migration of follicle dermal papilla cells, promotion of hair follicle neogenesis, promotion of hair growth, promotion of transition of hair from the telogen phase to the anagen phase, or an effect of maintaining the anagen phase of hair. Accordingly, the composition may have effects of preventing hair loss, promoting hair growth, and promoting hair growth of hair.

In one embodiment, the hair loss may be alopecia areata, androgenetic alopecia, postmenopausal alopecia, female pattern alopecia, seborrheic alopecia, alopecia pityroides, senile alopecia, chemotherapy-induced alopecia, radiation-induced alopecia, alopecia caused by trichotillomania, or postpartum alopecia.

In one embodiment, the composition may have antibacterial activity against bacteria and/or fungi.

In one embodiment, the composition may increase germinative layer cells, inner root sheath cells, outer root sheath cells, inner layer cells of hair follicles, medulla cells, or cortex/cuticle cells in hair follicles.

In one embodiment, the composition may activate a glycolysis pathway or autophagy.

In one embodiment, the composition may increase expression of anti-apoptotic genes, glycolytic enzyme genes, or autophagy-related genes in hair follicles.

In one embodiment, the composition may increase expression of β-catenin, c-myc, ERK1/2, AVEN, Ctsb, Ctsd, Lamp1, Sh3glb1, Dapk3, cathepsin, Hif1a, HRas, Ppp2ca, MCL1, GLUT1, HK2, or PKM2.

In one embodiment, the composition may decrease expression of ATF4, Gadd45, or PARP1 in hair follicles.

In one embodiment, the composition may have effects of moisture regulation, skin barrier restoration, or anti-inflammatory activity in skin tissue, particularly the scalp.

In one embodiment, the composition may be a topical composition, preferably a skin topical composition, and the skin topical composition is a collective term including any composition applied to the exterior of the skin, and may include cosmetics and pharmaceuticals in various formulations.

As used herein, the term “hair loss” refers to a phenomenon in which hair falls out from the scalp or a condition in which hair becomes sparse or thin. The term “prevention of hair loss” refers to preventing or inhibiting hair loss as described above, and the term “promotion of hair growth” includes not only promotion of generation of new hair but also promotion of healthy growth of existing hair.

As used herein, the term “apoptosis” is used in a broad sense and generally refers to regulated or programmed cell death in mammals accompanied by one or more characteristic cellular changes, including cytoplasmic shrinkage, loss of plasma membrane microvilli, nuclear fragmentation, degradation of chromosomal DNA, or loss of mitochondrial function. Apoptosis in hair follicles shortens the duration of the anagen phase and promotes premature entry into the catagen phase, thereby inhibiting hair growth and resulting in a reduction in hair follicle size.

The pharmaceutical composition of the present invention may further comprise, as an active ingredient, a known hair loss treatment in addition to chia seed mucilage, and may be used in combination with other known treatments for hair loss.

As used herein, the term “prevention” refers to any action that inhibits or delays occurrence, progression, or recurrence of hair loss by administration of the pharmaceutical composition according to the present invention, and the term “treatment” refers to any action that ameliorates or beneficially alters symptoms of a hair loss disease by administration of the composition of the present invention. Those skilled in the art to which the present invention pertains may determine exact criteria for diseases effectively treated by the present composition and evaluate degrees of improvement, enhancement, and treatment by referring to data presented by organizations such as the Korean Medical Association.

As used herein, the term “therapeutically effective amount” refers to an amount effective for preventing or treating a target disease when used in combination with the active ingredient of the present invention. The therapeutically effective amount of the composition of the present invention may vary depending on various factors, such as method of administration, target site, and condition of a patient. Accordingly, when used in humans, dosage should be determined at an appropriate level in consideration of both safety and efficacy. It is also possible to estimate an amount for human use from an effective amount determined through animal experiments. Considerations for determining an effective amount are described, for example, in Hardman and Limbird, eds., Goodman and Gilman's The Pharmacological Basis of Therapeutics, 10th ed. (2001), Pergamon Press; and E.W. Martin ed., Remington's Pharmaceutical Sciences, 18th ed. (1990), Mack Publishing Co.

The pharmaceutical composition of the present invention is administered in a pharmaceutically effective amount. As used herein, the term “pharmaceutically effective amount” refers to an amount sufficient to treat a disease at a reasonable benefit/risk ratio applicable to medical treatment without causing side effects. An effective dosage level may be determined depending on factors including a patient’s health condition, type of hair loss, cause and severity of hair loss, activity of a drug, sensitivity to the drug, method of administration, time of administration, route of administration, rate of excretion, duration of treatment, and drugs used in combination or concurrently, as well as other factors well known in the medical field. The composition of the present invention may be administered as a single therapeutic agent or in combination with other therapeutic agents, and may be administered sequentially or simultaneously with conventional therapeutic agents, and may be administered as a single dose or multiple doses. Considering all of the above factors, it is important to administer an amount that achieves maximum effect with a minimum amount without side effects, and such amount may be readily determined by those skilled in the art.

The pharmaceutical composition of the present invention may comprise a carrier, diluent, excipient, or a combination of two or more thereof, which are commonly used in biological formulations. As used herein, the term “pharmaceutically acceptable” refers to having no toxic properties to cells or humans exposed to the composition. The carrier is not particularly limited as long as it is suitable for delivery of the composition in vivo, and examples thereof include compounds described in The Merck Index, 13th ed., Merck & Co., Inc., saline, sterile water, Ringer’s solution, buffered saline, dextrose solution, maltodextrin solution, glycerol, ethanol, or a mixture of one or more thereof, and other conventional additives such as antioxidants, buffers, and bacteriostatic agents may be further added as necessary. In addition, formulations such as aqueous solutions, suspensions, emulsions, pills, capsules, granules, or tablets may be prepared by additionally adding diluents, dispersants, surfactants, binders, and lubricants. Furthermore, formulations may be prepared appropriately according to each disease or ingredient by methods known in the art or methods disclosed in Remington's Pharmaceutical Science (Mack Publishing Company, Easton PA, 18th, 1990).

In one embodiment, the pharmaceutical composition may be formulated for oral administration or parenteral administration depending on a route of administration. In the case of formulations for parenteral administration, the composition of the present invention may be formulated into injections, creams, lotions, topical agents, oils, moisturizers, gels, aerosols, or nasal inhalants by methods known in the art. These formulations are described in references generally known in pharmaceutical chemistry (Remington's Pharmaceutical Science, 15th Edition, 1975, Mack Publishing Company, Easton, Pennsylvania 18042, Chapter 87: Blaug, Seymour).

When the pharmaceutical composition of the present invention is used as a skin topical pharmaceutical composition, it may be prepared and used as a skin topical agent having effects of preventing hair loss, promoting hair growth, and improving scalp conditions in the form of a cream, gel, patch, spray, ointment, plaster, lotion, liniment, paste, or cataplasm, but is not limited thereto.

As used herein, the term “administration” refers to providing a predetermined substance to an individual or patient by any appropriate method, and depending on a desired method, may be parenteral administration (for example, application to the skin in the form of a topical formulation) or oral administration. Dosage may vary depending on body weight, age, sex, health condition, diet, time of administration, method of administration, excretion rate, and severity of a disease.

As used herein, the term “subject” or “individual” refers to all animals including humans, monkeys, cattle, horses, sheep, pigs, chickens, turkeys, quails, cats, dogs, mice, rats, rabbits, or guinea pigs, which have developed or may develop hair loss, and the above diseases may be effectively prevented or treated by administering the pharmaceutical composition of the present invention to the subject. The pharmaceutical composition of the present invention may be administered in combination with existing therapeutic agents.

The pharmaceutical composition of the present invention may further comprise pharmaceutically acceptable additives, and examples of the pharmaceutically acceptable additives include starch, pregelatinized starch, microcrystalline cellulose, lactose, povidone, colloidal silicon dioxide, calcium hydrogen phosphate, lactose, mannitol, syrup, gum arabic, pregelatinized starch, corn starch, powdered cellulose, hydroxypropyl cellulose, Opadry, sodium starch glycolate, carnauba wax, synthetic aluminum silicate, stearic acid, magnesium stearate, aluminum stearate, calcium stearate, sucrose, dextrose, sorbitol, and talc, but are not limited thereto. The pharmaceutically acceptable additives according to the present invention are preferably included in an amount of 0.1 parts by weight to 90 parts by weight based on the composition, but are not limited thereto.

In one aspect, the present invention relates to a cosmetic composition for preventing hair loss or promoting hair growth, comprising chia seed mucilage.

In one embodiment, the chia seed mucilage may be chia seed mucilage prepared by a method comprising mixing chia seeds with distilled water and stirring; collecting mucilage; and filtering.

In one embodiment, the composition may comprise a gel comprising chia seed mucilage.

In one embodiment, the gel comprising chia seed mucilage may be a chia seed mucilage polysaccharide (chia seed mucilage polysaccharides, CSMP) gel prepared by a method comprising mixing and stirring chia seed mucilage with PVA (poly(vinyl alcohol)); adding and stirring D-sorbitol and glycerin; and performing UV treatment.

In one embodiment, the composition may further comprise chia seed oil (chia seed oil, CSO).

In one embodiment, the composition may comprise a gel comprising chia seed mucilage and a microencapsulation cream comprising chia seed oil.

In one embodiment, the microencapsulation cream may be a chia seed-based microencapsulation (Chia Seed-based Microencapsulation, CSMi) cream prepared by a method comprising mixing and stirring SDS (sodium dodecyl sulfate) with chia seed oil; adding and stirring a gel comprising chia seed mucilage; and performing UV treatment.

In one embodiment, the cosmetic composition may be formulated as a skin lotion, gel, aqueous liquid, cream, essence, oil-in-water (O/W) type formulation, or water-in-oil (W/O) type formulation, and may be a hair tonic, hair conditioner, hair essence, hair lotion, hair nourishing lotion, hair shampoo, hair rinse, hair treatment, hair cream, hair nourishing cream, hair moisture cream, hair massage cream, hair wax, hair aerosol, hair pack, hair nourishing pack, hair soap, hair cleansing foam, hair oil, hair drying agent, hair preserving treatment agent, hair dye, hair waving agent, hair bleaching agent, hair gel, hair glaze, hair dressing, hair lacquer, hair moisturizer, hair mousse, or hair spray.

In one embodiment, the cosmetic composition may be prepared in any formulation commonly manufactured in the art, for example, solutions, suspensions, emulsions, pastes, gels, creams, lotions, powders, soaps, surfactant-containing cleansers, oils, powder foundations, emulsion foundations, wax foundations, and sprays, but is not limited thereto.

The cosmetic composition of the present invention may be prepared by comprising a cosmetically effective amount of the active ingredients of the present invention and a cosmetically acceptable carrier.

As used herein, the term “cosmetically effective amount” refers to an amount sufficient to achieve the hair damage prevention efficacy of the composition of the present invention described above.

The appearance of the cosmetic composition comprises a cosmetically or dermatologically acceptable medium or base. The cosmetic composition may be provided in any formulation suitable for topical application, for example, solutions, gels, solids, anhydrous paste products, emulsions obtained by dispersing an oil phase in an aqueous phase, suspensions, microemulsions, microcapsules, microgranules, or ionic (liposomal) or non-ionic vesicular dispersions, or may be provided in the form of creams, skins, lotions, powders, ointments, sprays, or concealer sticks. These compositions may be prepared by conventional methods in the art. The composition according to the present invention may also be used in the form of a foam or an aerosol composition further comprising a compressed propellant.

When the formulation of the cosmetic composition of the present invention is a paste, cream, or gel, animal fibers, plant fibers, waxes, paraffin, starch, tragacanth, cellulose derivatives, polyethylene glycol, silicone, bentonite, silica, talc, or zinc oxide may be used as carrier components.

When the formulation of the cosmetic composition of the present invention is a powder or spray, lactose, talc, silica, aluminum hydroxide, calcium silicate, or polyamide powder may be used as carrier components, and particularly in the case of a spray, a propellant such as chlorofluorohydrocarbon, propane/butane, or dimethyl ether may be further included.

When the formulation of the cosmetic composition of the present invention is a solution or emulsion, a solvent, solubilizing agent, or emulsifying agent may be used as a carrier component, and examples thereof include water, ethanol, isopropanol, ethyl carbonate, ethyl acetate, benzyl alcohol, benzyl benzoate, propylene glycol, 1,3-butylene glycol oil, glycerol aliphatic esters, polyethylene glycol, or fatty acid esters of sorbitan.

When the formulation of the cosmetic composition of the present invention is a suspension, liquid diluents such as water, ethanol, or propylene glycol; suspending agents such as ethoxylated isostearyl alcohol, polyoxyethylene sorbitol esters, and polyoxyethylene sorbitan esters; and microcrystalline cellulose, aluminum metahydroxide, bentonite, agar, or tragacanth may be used as carrier components.

When the formulation of the cosmetic composition of the present invention is a surfactant-containing cleanser, aliphatic alcohol sulfates, aliphatic alcohol ether sulfates, sulfosuccinic acid monoesters, isethionates, imidazolinium derivatives, methyl taurate, sarcosinates, fatty acid amide ether sulfates, alkylamidobetaines, aliphatic alcohols, fatty acid glycerides, fatty acid diethanolamides, vegetable oils, lanolin derivatives, or ethoxylated glycerol fatty acid esters may be used as carrier components.

In one embodiment, the cosmetic composition according to the present invention may further comprise, in addition to the above-described composition, functional additives and components commonly included in cosmetic compositions. Examples of the functional additives may include ingredients selected from the group consisting of water-soluble vitamins, oil-soluble vitamins, polymer peptides, polymer polysaccharides, sphingolipids, and seaweed extracts.

The cosmetic composition of the present invention may further comprise, as necessary, components commonly included in cosmetic compositions in addition to the above-described composition. Examples of such components include oily components, moisturizers, emollients, surfactants, organic and inorganic pigments, organic powders, ultraviolet absorbers, preservatives, disinfectants, antioxidants, plant extracts, pH adjusters, alcohols, colorants, fragrances, blood circulation promoters, cooling agents, antiperspirants, and purified water.

In one aspect, the present invention relates to a method for preparing a chia seed mucilage polysaccharide (chia seed mucilage polysaccharides, CSMP) gel, comprising: mixing chia seeds with distilled water and stirring; collecting chia seed mucilage; mixing and stirring the chia seed mucilage with PVA; adding and stirring D-sorbitol and glycerin; and performing UV treatment.

In one aspect, the present invention relates to a method for preparing a chia seed-based microencapsulation (Chia Seed-based Microencapsulation, CSMi) cream, comprising: mixing and stirring SDS with chia seed oil; adding and stirring a chia seed mucilage polysaccharide gel; and performing UV treatment.

In one aspect, the present invention relates to a method for preventing or treating hair loss, comprising administering an effective amount of a pharmaceutical composition comprising chia seed mucilage to a subject in need of prevention or treatment of hair loss.

In one embodiment, the “subject” or “individual” to be administered according to the method may be any animal including a human who has developed or may develop hair loss, and may specifically be an individual suffering from various hair loss diseases such as androgenetic alopecia, alopecia areata, or telogen effluvium.

In one embodiment, the method may comprise administering the composition to the scalp or hair follicles of the subject to promote hair follicle neogenesis, induce transition of hair from the telogen phase to the anagen phase, and maintain the anagen phase.

In one embodiment, the method of administration may be a method of directly applying, spraying, or massaging the pharmaceutical composition onto the scalp of the subject in the form of a skin topical formulation. In particular, when the composition is in the form of a chia seed-based microencapsulation (CSMi) cream, microcapsules having a particle size of 500 to 5000 nm may maximize delivery efficiency of active ingredients into the dermis and inside hair follicles.

In one embodiment, the method may comprise inducing proliferation of germinative layer cells, inner root sheath cells, and outer root sheath cells within hair follicles through administration.

In one embodiment, the method may regulate biological signaling pathways within hair follicle tissue of the subject. Specifically, the administration may increase expression of β-catenin, c-myc, ERK1/2, AVEN, cathepsin, or glycolytic enzymes such as GLUT1, HK2, and PKM2, while simultaneously decreasing expression of ATF4, Gadd45, or PARP1 associated with apoptosis or endoplasmic reticulum stress, thereby inhibiting apoptosis and promoting hair growth.

In one embodiment, the dosage or “therapeutically effective amount” of the composition may be appropriately determined by those skilled in the art depending on the condition of the subject, severity of hair loss, and route of administration. The method may comprise administering the composition alone or in combination with other known hair loss treatments or therapeutic methods.

The present invention will be described in further detail through the following examples. However, these examples are provided for illustrative purposes only and are not intended to limit the scope of the invention in any way.

### Example

### Example 1. Preparation of a Cream Comprising Chia Seed-Derived Mucilage and Oil

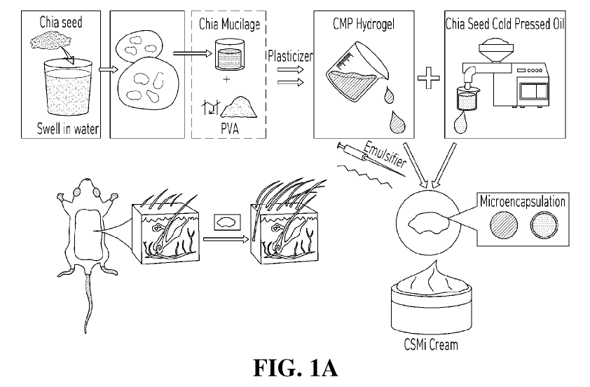

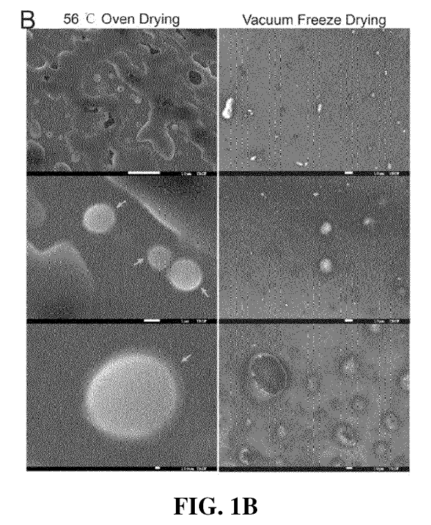

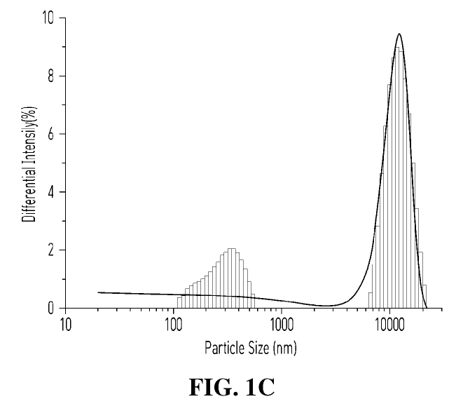

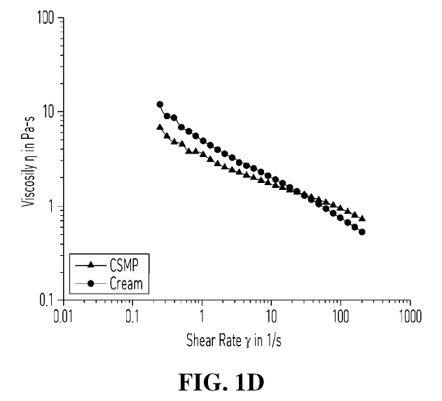

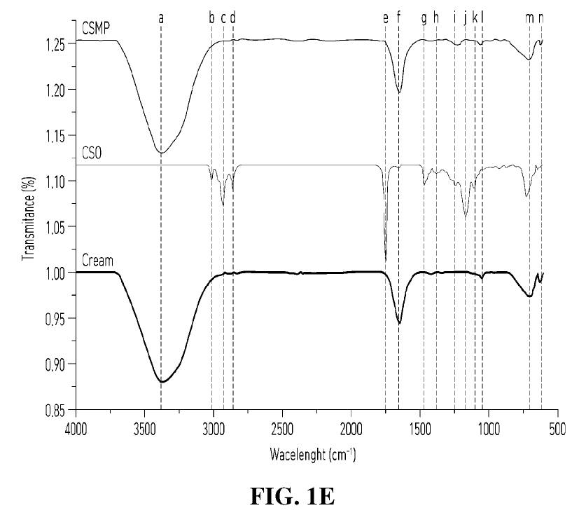

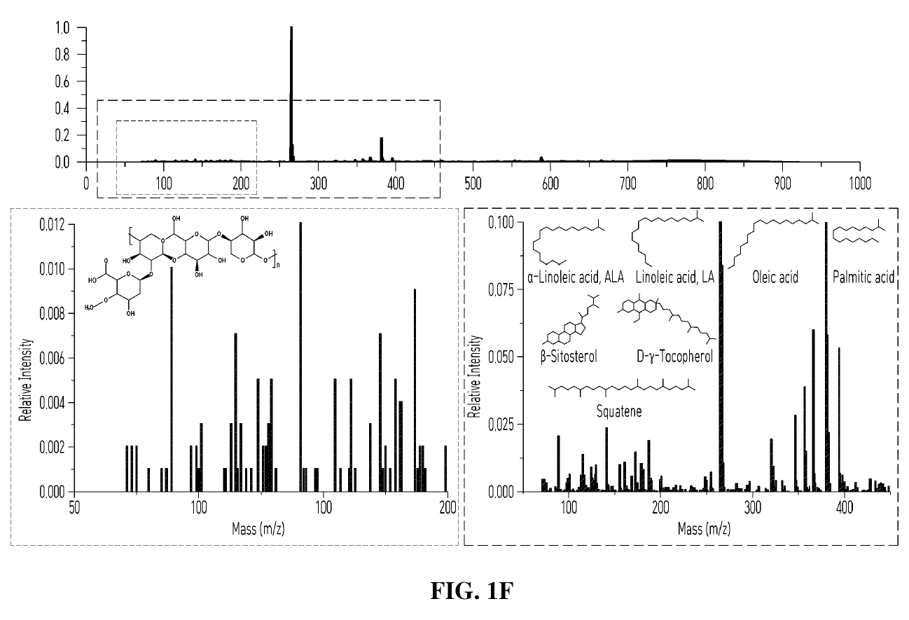

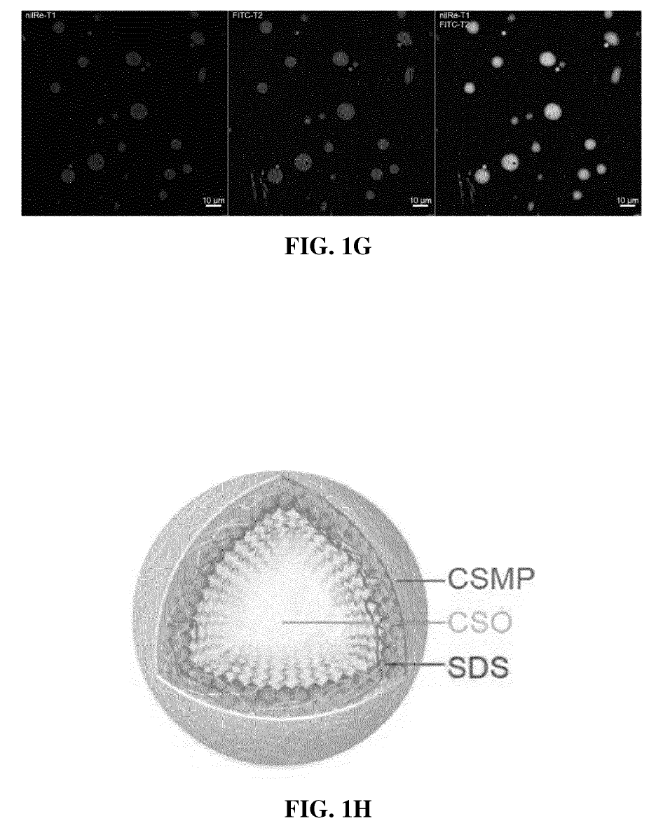

A chia seed-based microencapsulation (Chia Seed-based Microencapsulation, CSMi) cream was prepared using a CSMP (chia seed mucilage polysaccharides) gel prepared from chia seed mucilage and chia seed oil (chia seed oil, CSO) (FIG. 1A). Specifically, chia seeds and distilled water were placed in a glass beaker at a weight ratio of 1:30 and continuously stirred at room temperature for 3 hours using a magnetic stirrer to allow mucilage to be released from the chia seeds. The mixture was centrifuged at 4000 RPM for 1 hour, and the upper mucilage layer was collected and filtered through a 200-mesh cheesecloth. In addition, the swollen chia seeds in the lower layer were pressed and filtered through a 200-mesh bag to maximize recovery of the mucilage. The extracted chia seed mucilage was freeze-dried and stored at room temperature.

To prepare a gel, 0.6 g of the freeze-dried mucilage was mixed with 8 mL of distilled water, and 12 mL of a 5% (w/v) PVA (poly(vinyl alcohol)) solution (molecular weight 146,000 to 186,000, degree of hydrolysis ≥99%) was added, followed by stirring at 37°C until completely dissolved. Thereafter, 0.375 g of D-sorbitol and 0.375 g of glycerin were added and stirred at 500 RPM for 1 hour to obtain a solution having final concentrations of 3% (w/v) CM, 3% (w/v) PVA, 1.5% (w/v) sorbitol, and 1.5% (v/v) glycerin. The prepared CSMP solution was exposed to UV light (254 nm) for 30 minutes to prepare a 3% CSMP gel, which was stored at 4°C.

To prepare a CSMi cream, direct emulsification of CSO using the CSMP gel under elevated temperature and high shear conditions was attempted, but phase separation occurred upon standing. Accordingly, SDS (sodium dodecyl sulfate) was introduced to obtain a stable emulsion. Specifically, 20 mL of chia seed oil (CSO) was placed in a beaker, and 4 mL of a 10% UltraPure™ SDS Solution (15553027, Invitrogen (USA)) was added and stirred using a magnetic stirrer until the mixture became opaque white to form an emulsion. To the emulsified chia seed oil, 20 mL of the above-prepared 3% CSMP gel was added and stirred for 30 minutes to homogenize the mixture. The prepared cream was exposed to UV light (254 nm) to induce crosslinking and sterilization and then stored at 4°C.

At this time, emulsification degrees were compared using various concentrations of SDS. As a result, an SDS concentration of 1% formed a less stable emulsion, and 0.2% SDS exhibited limited emulsification effects. When the final SDS concentration was 0.1%, phase separation occurred during storage, and samples containing 0.5% SDS exhibited partial emulsification, whereas an SDS concentration of 2% provided optimal emulsification (data not shown). In addition, results of further examination of various mixing ratios of CSMP gel to CSO showed that the 0.1% SDS group exhibited insufficient emulsification of CSO and phase separation, whereas the 1% SDS group formed stable creams at all ratios. Further, in the 0.5% SDS group, a stable cream was formed when CSMP accounted for one-third of the formulation, but phase separation occurred when the oil fraction exceeded half, suggesting that although the CSMP gel has emulsifying capability, stable emulsification is difficult when used alone.

### Example 2. Characterization of the Chia Seed-Based Microencapsulation Cream

### 2-1. Structural Analysis

Structural characteristics of the CSMi cream prepared in Example 1 were examined using SEM. Specifically, oven-dried samples and vacuum freeze-dried samples of the CSMi cream were each fixed onto a metal platform using a conductive adhesive, and images at magnifications of 1,000× to 10,000× were captured using a field emission scanning electron microscope (JSM-7610F-Plus, JEOL, Japan) with an accelerating voltage set to 10 kV or 15 kV.

As a result, the CSMi cream was observed to contain numerous particles having smooth surfaces and distinct spherical shapes (FIG. 1B). In the oven-dried samples, the spherical particles were surrounded by a uniformly distributed mist-like coating, which was presumed to be residual materials evaporated during the drying process. In the case of the vacuum freeze-dried samples, some larger particles were ruptured due to internal–external pressure differences, revealing internal structures and confirming the presence of a solid shell encapsulating the contents. In addition, a uniform network structure corresponding to glassy CSMP was observed in the background of the vacuum freeze-dried samples.

### 2-2. Particle Size Analysis

To determine the particle size of microcapsules in the CSMi cream prepared in Example 1, the sample was diluted 20-fold with water and measured using a Particle Size & Zeta Potential Analyzer (ELS-Z1000, Otsuka Electronics). As a result, the average size of the microcapsules in the CSMi cream was determined to be 2107.7 nm (FIG. 1C). The zeta potential was measured to be −101.377 mV, confirming strong stability.

### 2-3. Viscosity Analysis

Viscosities of the CSMP gel and the CSMi cream prepared in Example 1 were measured using a rheometer (MCR 302e, Anton Paar) with a parallel plate geometry (PP25) having a diameter of 25 mm and a gap size of 1.0 mm, at shear rates ranging from 0.1 s⁻¹ to 200 s⁻¹.

As a result, both the CSMP gel and the CSMi cream exhibited typical shear-thinning behavior characteristic of non-Newtonian fluids. The overall viscosity of the cream was significantly higher than that of the CSMP gel, particularly at low shear rates (FIG. 1D). This is likely due to a denser network structure of molecules or particles, or a higher content of thickeners or oil-phase components. Accordingly, the CSMP gel is suitable for products requiring high fluidity and easy spreading, whereas the cream having higher viscosity and greater structural stability is more suitable for moisturizing and protective skin care applications.

### 2-4. Chemical Composition Analysis

Chemical compositions of chia seed oil (CSO), the CSMP gel, and the CSMi cream prepared in Example 1 were analyzed by FTIR spectroscopy using a spectrometer (VERTEX 70, Bruker, USA) with 16 scans at a resolution of 4 cm⁻¹ over a scanning range of 4000 to 500 cm⁻¹.

As a result, the CSMP gel exhibited a broad peak at 3375.39 cm⁻¹ (a), attributed to O–H stretching typically observed in polysaccharides, indicating the presence of abundant hydroxyl groups. A peak at 1644.02 cm⁻¹ (f), corresponding to amide I (C=O stretching), suggested the presence of protein moieties associated with galactomannan (FIG. 1E). The symmetric COO⁻ peak at 1411.71 cm⁻¹ (between g and h) indicated carboxyl groups of uronic acids, and the absorbance at 1047.90 cm⁻¹ (l) represented C–O–C 1→4 glycosidic ring vibrations characteristic of polysaccharides. A peak at 695.05 cm⁻¹ (m) corresponded to β-anomeric C–H deformation, indicating the presence of glycosidic linkages containing glucopyranose and xylopyranose units. A peak at 618.35 cm⁻¹ (n) indicated the crystalline nature of the gel, suggesting a certain degree of crystallinity in the sample.

In the case of CSO, the peak at 3011.585 cm⁻¹ (b) was attributed to C–H stretching of cis-olefinic groups (–HC=CH–) dependent on conjugated C=C bonds in fatty acids, indicating a high degree of unsaturation including a substantial proportion (~55%) of α-linolenic acid. Bands at 2930.49 cm⁻¹ (c) and 2855.98 cm⁻¹ (d) corresponded to C–H stretching vibrations of methyl and methylene groups, representing the lipid backbone. The peak observed at 1744.83406 cm⁻¹ (e) was associated with C=O stretching of ester groups. The band at 1457.73 cm⁻¹ (g) was attributed to CH₂ bending vibration, and the peak at 1372.26 cm⁻¹ (h) corresponded to a combination of deformation modes of methyl and methylene groups. The peak at 1238.57 cm⁻¹ (i) represented C–O stretching in carbohydrate-related structures, and the band at 1164.06 cm⁻¹ (j) corresponded to asymmetric stretching of ester groups (C–C(=O)–O). The peak at 1100.50 cm⁻¹ (k) was assigned to symmetric C–O–C stretching characteristic of triglycerides and esters. At 723.544145 cm⁻¹ (between l and m), rocking and out-of-plane deformation of methylene groups representing cis-disubstituted olefins were observed, confirming the unsaturated characteristics of chia seed oil.

In the case of the CSMi cream, which is a mixture of the CSMP gel and CSO, the spectrum exhibited strong bands consistent with the CSMP gel, while intensities of bands corresponding to CSO were reduced, which was presumed to be due to the encapsulation effect.

### 2-5. LC–MS Analysis

Chia seed oil (CSO), the CSMP gel, and the CSMi cream prepared in Example 1 were analyzed using a high-resolution liquid chromatography mass spectrometer (Exploris 240, ThermoFisher). As a result, within a major range (red box) corresponding to CSMP gel units, a fragment ion at m/z 179.06 corresponded to demethylated 4-O-methyl-α-D-glucopyranosyl, and fragment ions at m/z 176.95 and 160.84 corresponded to 4-O-methyl-α-D-glucopyranosyl fragments lacking hydroxyl groups (FIG. 1F). In addition, a major range corresponding to CSO (green box) showed that α-linolenic acid (ALA) was prominent as a protonated ion at m/z 279.23 (FIG. 1F). Taken together, these analytical results confirmed that the CSMi cream contained nutritional components derived from both the CSMP gel and CSO.

### 2-6. Analysis of Microcapsule Structure of the Cream

To analyze the microcapsule structure of the CSMi cream by imaging, 25 μL of Nile Red (NilRe-T1) (0.57 mg/mL) was added to 10 mL of CSO and mixed for 5 minutes, followed by addition of SDS to form an emulsion. Separately, 1.5 mg of FITC-T2 (fluorescein 5(6)-isothiocyanate) was added to a CSMP solution and stirred for 10 minutes. The two fluorescently labeled components were combined and used to prepare the CSMi cream according to the same method as in Example 1, followed by stirring for 30 minutes.

Thereafter, confocal microscopy analysis was performed using a confocal microscope (LSM 980, Carl Zeiss). Fluorescence of Nile Red was excited with a 561 nm laser and detected in an emission range of 580 to 650 nm, and FITC fluorescence was excited with a 488 nm laser and detected in an emission range of 500 to 550 nm. Imaging data were analyzed using Zeiss Zen software.

As a result, the microcapsule structure of the CSMi cream was observed to be stable and uniformly distributed, and colocalization between the oil phase and the polysaccharide phase was observed (FIG. 1G). Accordingly, it was confirmed that during synthesis of the CSMi cream, the CSMP gel served as a wall material that encapsulated CSO emulsified by SDS (FIG. 1H).

### Example 3. In Vitro Effect Analysis of the Chia Seed-Based Microencapsulation Cream

### 3-1. Cytotoxicity Analysis

To evaluate cytotoxicity of the CSMi cream, the CSMP gel and the CSMi cream were each dissolved at various concentrations in DMEM without fetal bovine serum (FBS) or growth factors. Human follicle dermal papilla cells (HFDPCs, C-12071, PromoCell, Germany), cultured in Follicle Dermal Papilla Cell Growth Medium, were seeded at 5,000 cells per well in a 96-well plate and treated with the prepared samples. After 24 hours, an MTT assay was performed.

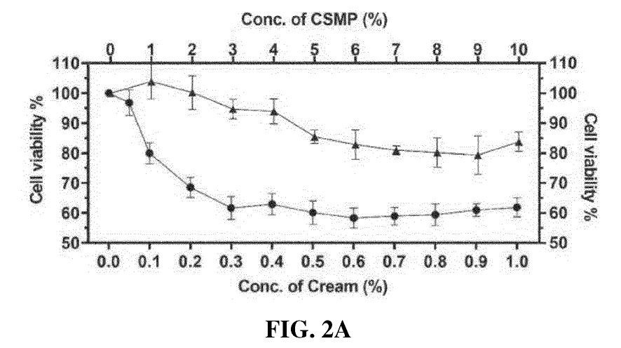

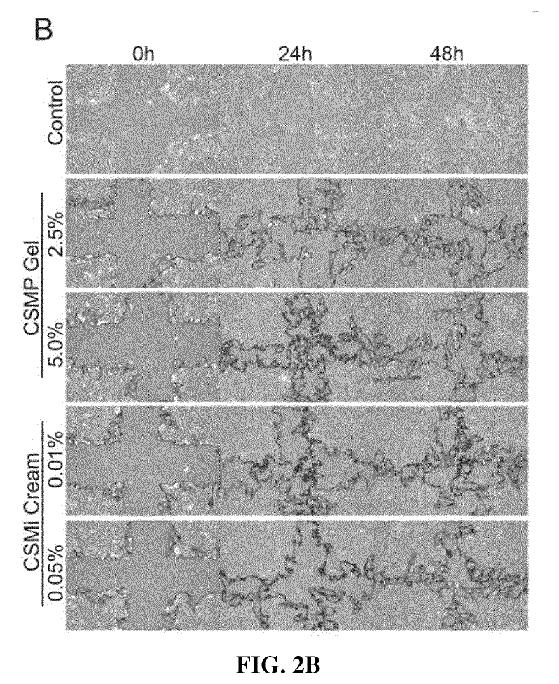

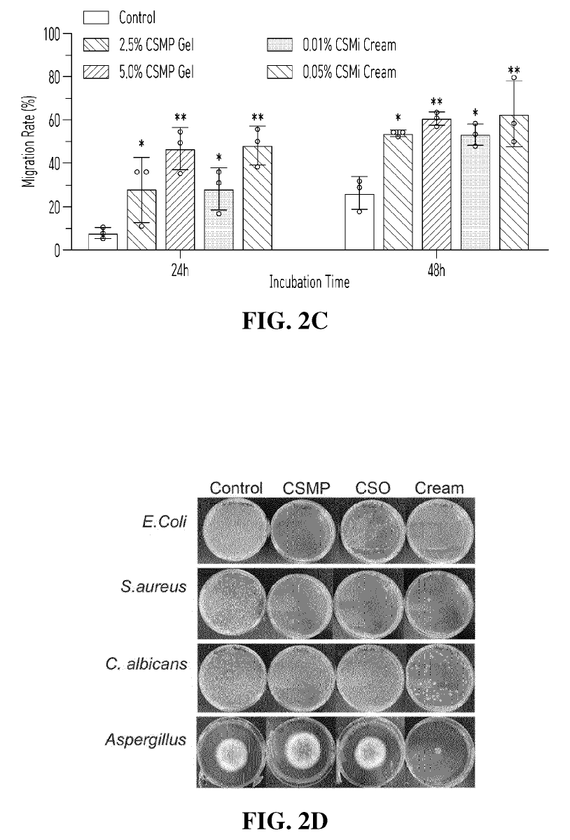

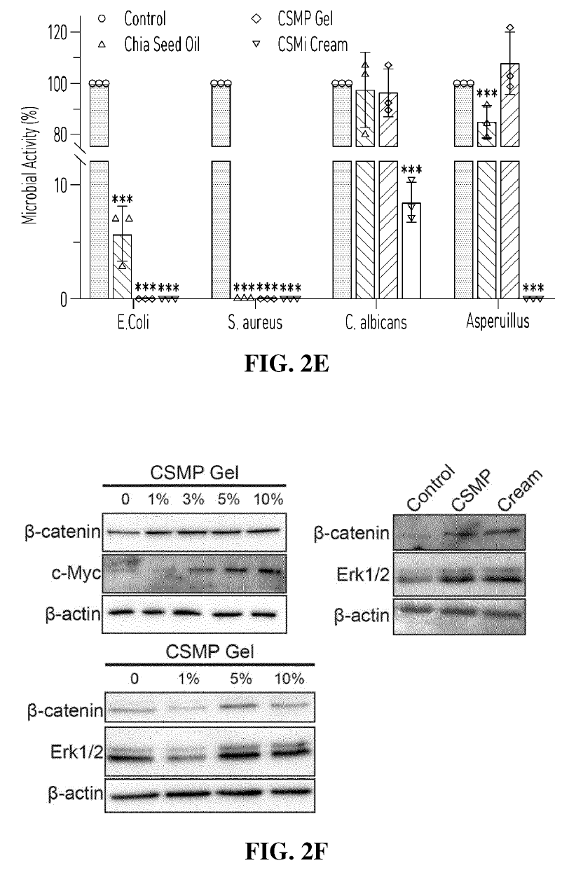

As a result, the CSMP gel exhibited cell viability of 80% or higher at concentrations of 1 to 10%, indicating excellent biocompatibility (FIG. 2A). In contrast, the CSMi cream exhibited a decrease in cell viability as concentration increased (FIG. 2A), confirming the need to select an appropriate concentration to minimize cytotoxicity.

### 3-2. Cell Migration Analysis

Since migration of HFDPCs is closely associated with the hair growth cycle, effects of the CSMi cream on cell migration were evaluated. Specifically, HFDPCs were seeded at 2 × 10⁵ cells per well in a 6-well plate and cultured for 24 hours until reaching 60 to 80% confluence. A scratch was then created on the cell monolayer surface, and detached cells were removed. Thereafter, 2 mL of DMEM without FBS or growth factors containing various concentrations of the CSMP gel or the CSMi cream was added to each well.

Images of the scratch area were captured at predetermined time intervals, and the area of the scratch was quantified using ImageJ software. Cell migration ability was analyzed by calculating a closure ratio of the scratch area.

As a result, both the CSMP gel and the CSMi cream significantly promoted cell migration. In particular, 5.0% CSMP gel and 0.05% CSMi cream exhibited the most pronounced effects within 48 hours (FIGS. 2B and 2C).

### 3-3. Antimicrobial Activity Analysis

To evaluate antimicrobial properties of the CSMi cream, antimicrobial effects against bacteria and fungi were analyzed. Specifically, Escherichia coli (E. coli), Staphylococcus aureus (S. aureus), and Candida albicans (C. albicans) were diluted to an initial concentration of 10⁷ CFU/mL, and Aspergillus fumigatus was diluted to an initial concentration of 10⁴ cells/mL, and each was inoculated into liquid culture media. Thereafter, 100 μL each of the CSMP gel, CSO, and the CSMi cream was added to the culture media and incubated at 37°C for 24 hours.

After incubation, samples of E. coli, S. aureus, and C. albicans were further diluted 10,000-fold, whereas A. fumigatus samples were not diluted, and 100 μL of each was spread onto solid culture media and incubated at 37°C for 16 hours. Thereafter, colony-forming units (CFUs) were counted and quantitatively analyzed using ImageJ software.

As a result, both the CSMP gel and CSO exhibited strong antibacterial effects but showed weaker effects against fungi (FIGS. 2D and 2E). In contrast, the CSMi cream exhibited excellent antimicrobial activity against all tested microorganisms and showed the most potent effects, completely inhibiting E. coli, S. aureus, and A. fumigatus at the same concentration (FIGS. 2D and 2E).

### 3-4. Molecular Mechanism Analysis of Hair Growth

β-Catenin, which plays a critical role in molecular mechanisms of hair growth, is a core component of the Wnt signaling pathway and is essential for development, regeneration, and the overall hair growth cycle of hair follicles (HFs). Accordingly, expression of β-catenin in HFDPCs treated with the CSMP gel or the CSMi cream was examined by western blot analysis.

As a result, both the CSMP gel and the CSMi cream increased expression of β-catenin in HFDPCs (FIG. 2F). In addition, the CSMP gel activated c-myc, which regulates hair growth cycles in mice and promotes proliferation of matrix keratinocytes and differentiation of inner root sheath cells. Both the CSMP gel and the CSMi cream also increased expression of ERK1/2, which enhances activity of anti-apoptotic proteins (FIG. 2F).

Taken together, these results confirmed that the CSMP gel and the CSMi cream exhibit excellent cytocompatibility and antimicrobial properties, promote migration of dermal papilla cells, and exert hair growth effects by activating molecular mechanisms essential for hair growth.

### Example 4. In Vivo Hair Growth-Promoting Effects of the Chia Seed-Based Microencapsulation Cream

### 4-1. Analysis of Hair Growth-Promoting Effects

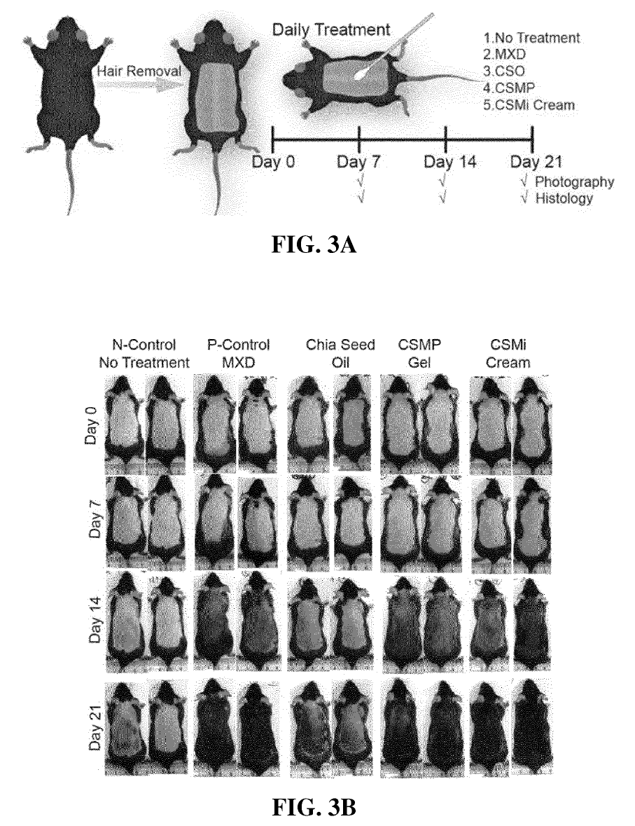

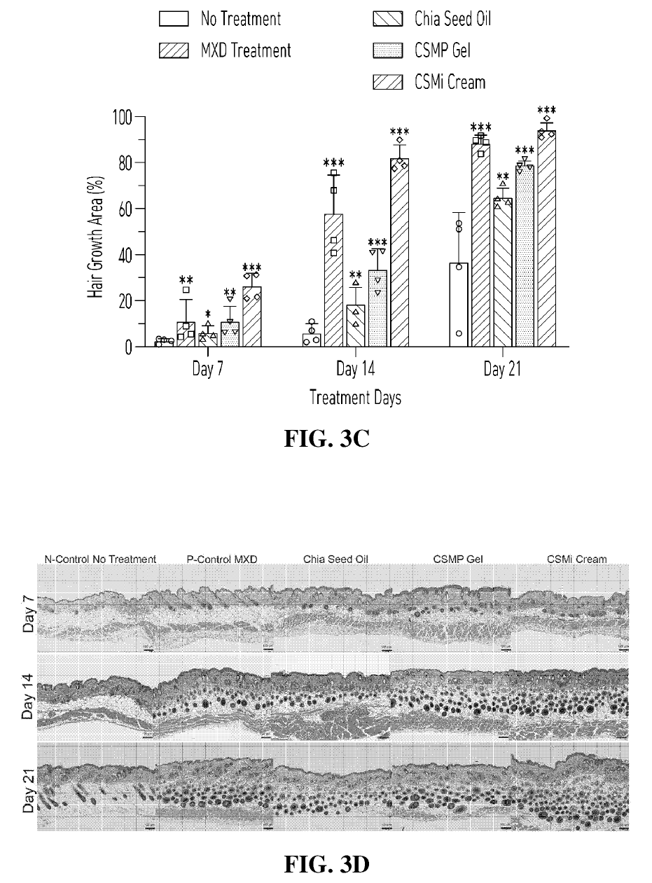

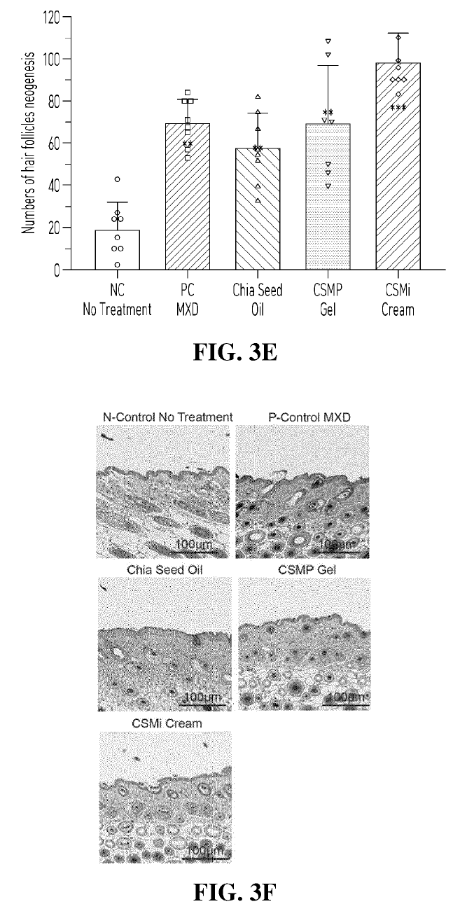

Hair growth-promoting effects of the CSMi cream in vivo were evaluated using a mouse depilation model. Specifically, 9-week-old female C57BL/6 mice (24–30 g) were divided into five groups: (1) a negative control group treated with PBS; (2) a positive control group treated with minoxidil; (3) a treatment group treated with CSO oil (topical application); (4) a treatment group treated with the CSMP gel; and (5) a treatment group treated with the CSMi cream. After anesthesia with isoflurane, dorsal regions of the mice were depilated, and minoxidil was applied at 5% per mouse, while CSO, the CSMP gel, and the CSMi cream were each applied at 200 μL per mouse to the depilated skin at a fixed time daily. Mice were anesthetized and photographed, and tissue samples were collected at 1, 2, and 3 weeks of the experiment (FIG. 3A).

As a result, the CSMP gel and the CSMi cream markedly accelerated initiation of the anagen phase (FIG. 3B). Unlike the CSO-treated group, which exhibited changes only at day 14, the minoxidil-treated group, the CSMP gel-treated group, and the CSMi cream-treated group showed changes in dorsal skin color as early as day 7 after depilation. At day 14, distinct hair growth was observed in the minoxidil-treated group, the CSMP gel-treated group, and the CSMi cream-treated group. In particular, in the CSMi cream-treated group, dorsal regions of most mice were almost completely covered with dark-colored hair (FIG. 3B). Quantitative analysis confirmed hair growth-promoting effects of the CSMP gel and the CSMi cream (FIG. 3C).

### 4-2. Histological Analysis

Tissues collected in Example 4-1 were preserved in a 4% neutral buffered formalin solution, embedded in paraffin, and sliced into 4 μm sections. The sections were stained with hematoxylin and eosin (H&E) and observed under an optical microscope to evaluate whether the CSMi cream accelerated transition of hair follicles (HFs) from the telogen phase to the anagen phase. Data were analyzed using Cellsens software and quantified using ImageJ software.

As a result, hair follicle neogenesis was observed in the CSMP gel-treated group and the CSMi cream-treated group at day 7, and at day 14, the number of newly generated HFs was markedly increased in the CSMi cream-treated group compared to other groups, showing complete structural regeneration of HFs (FIG. 3D). Quantitative analysis revealed that hair follicle neogenesis was significantly increased in the CSMi cream-treated group (FIG. 3E). More detailed histological analysis showed that, unlike the CSMi cream-treated group, the CSO- treated group exhibited thickened epidermis and abnormally enlarged sebaceous glands (FIG. 3F). This was presumed to be due to fatty acids in the oil disrupting the skin barrier and clogging pores, thereby stimulating excessive cell proliferation. These results indicate that the CSMi cream, which combines polysaccharides and oil, allows the polysaccharides to form a protective layer that reduces direct contact between the oil and the skin while simultaneously providing moisture regulation, skin barrier restoration, and anti-inflammatory effects, thereby promoting hair follicle neogenesis and providing safer and healthier skin care effects.

### Example 5. Analysis of Effects of the CSMi Cream on Regulation of Hair Follicle Heterogeneity

### 5-1. Single-Cell Transcriptome Analysis

To comprehensively investigate cell types and transcriptional profiles involved in transition of hair growth from the telogen phase to the anagen phase, single-cell libraries were prepared and gene expression analyses were performed for individual cells. Specifically, dorsal skin tissues of mice from a group treated with PBS for 14 days (untreated group) and a group treated with the CSMi cream for 14 days were harvested and minced. The tissues were treated with a digestion solution comprising 1 mg/mL collagenase I, 1 mg/mL collagenase IV, 1 mg/mL dispase, 0.125% trypsin-EDTA, 2 U/mL DNase, and DMEM/F12, followed by incubation at 37°C for 1 hour.

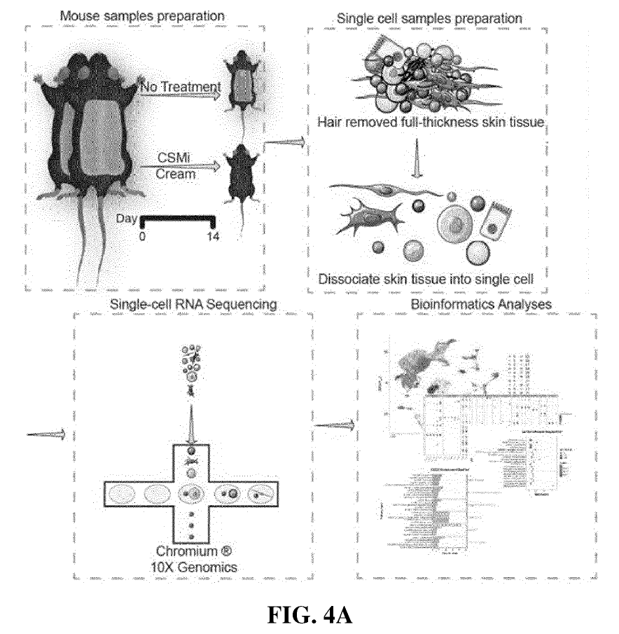

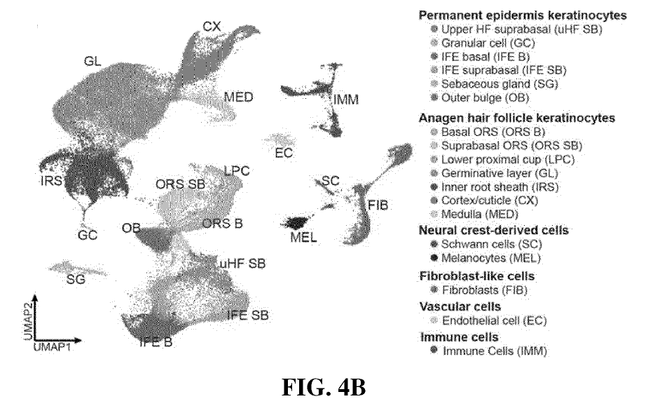

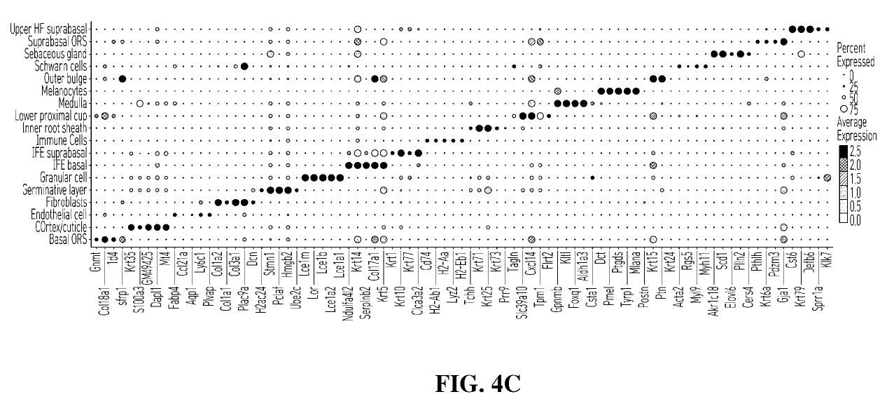

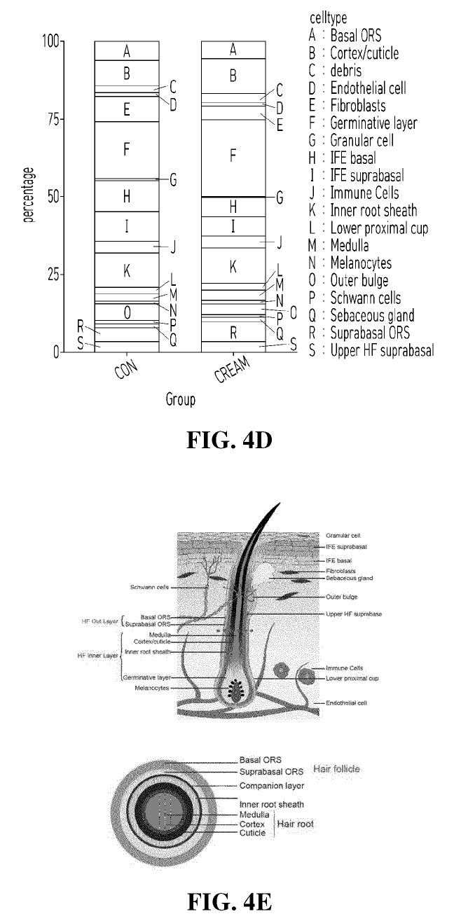

Thereafter, the tissues were pipetted to obtain a single-cell suspension and passed through a 40 μm cell strainer. Enzymatic digestion was terminated by adding DMEM/F12 containing 10% FBS, and the cells were centrifuged at 300×g for 5 minutes at 4°C, collected, and resuspended in 5 mL of cold PBS. The isolated single-cell suspensions were loaded into a droplet-based 10X Genomics Chromium system to capture 8,000 single cells, followed by cDNA amplification and library construction according to a standard protocol (FIG. 4A). The libraries were sequenced on an Illumina NovaSeq 6000 sequencing system (paired-end multiplex run, 150 bp) at LC-Bio Technology Co., Ltd. (Hangzhou, China) with a minimum depth of 20,000 reads per cell. Sequencing data were analyzed using CellRanger software (v7.0.0, 10X Genomics) to obtain gene expression information for each cell, and CellRanger outputs were loaded into Seurat software (v4.1.1) for dimensionality reduction, clustering, and scRNA-seq data analysis. As a result, a total of 81,982 cells, including 42,669 cells from the untreated group and 39,313 cells from the CSMi cream-treated group, were sequenced.

### 5-2. UMAP (Uniform Manifold Approximation and Projection) Embedding Analysis

UMAP embedding analysis was performed based on the sequencing results, and as a result, 18 distinct cell clusters were identified (FIG. 4B).

### 5-3. DEG Analysis

To classify cell types, differentially expressed genes (DEGs) were analyzed in each cluster identified in Example 5-2 and cross-referenced with known markers. As a result, specific genes represented various cell types within hair follicles and related tissues (FIG. 4C). For example, genes such as Lce1a and Lce2f were specific to suprabasal hair follicle cells and suprabasal interfollicular epidermal (IFE) cells. Lgr5 and Dcn were enriched in outer bulge cells, highlighting their stem cell-related functions. Melanocytes closely associated with pigmentation highly expressed Tyr and Mitf, while sebaceous gland cells expressed Elovl3 and Elovl6, indicating their roles in lipid metabolism. Inner root sheath cells were characterized by keratin family genes such as Krt25 and Krt27, whereas cortex/cuticle cells exhibited high expression of Krt35 and Krt85, consistent with their functions in hair shaft formation. Immune cells and fibroblasts expressed Cd74, H2-Ab1, Col1a1, and Col3a1, reflecting their biological roles. These gene expression patterns demonstrated functional diversity and cooperative mechanisms of cell types within the hair follicle ecosystem.

### 5-4. Cell Type Distribution Analysis

Analysis of cell type distributions in the untreated group and the CSMi cream-treated group showed that germinative layer cells were the most abundant cluster in both groups (18.78% and 23.91%, respectively). Inner root sheath cells were the second most abundant, accounting for 10.82% in the untreated group and 11.46% in the CSMi cream-treated group. Differences were observed in cortex/cuticle cells, which ranked third at 11.16% in the CSMi cream-treated group but ranked fifth at 8.23% in the untreated group (FIG. 4D). In addition, the proportion of outer root sheath (ORS) cells increased from 10.38% to 11.66% following CSMi cream treatment, and the proportion of inner layer cells increased from 40.12% to 50.79%. Among these, medulla cells, which constitute the innermost structure of the hair shaft and are often associated with visible hair growth characteristics, showed the most pronounced increase. Furthermore, based on physiological characteristics of the 18 cell subpopulations, the subpopulations were mapped onto a schematic diagram (FIG. 4E). These results indicate that the CSMi cream effectively promotes hair follicle regeneration.

### 5-5. Analysis of Hair Follicle Outer and Inner Layers

The outer layer of hair follicles includes critical subpopulations such as basal ORS and suprabasal ORS, which are essential for structural maintenance and proliferation of outer root sheath cells. In contrast, the inner layer of hair follicles comprises the medulla, cortex/cuticle, inner root sheath, and germinative layer, all of which contribute to formation and differentiation of hair shaft and root structures. Since the outer and inner layers provide essential cellular and molecular foundations for hair follicle development and regeneration, the outer and inner layers were further classified and annotated.

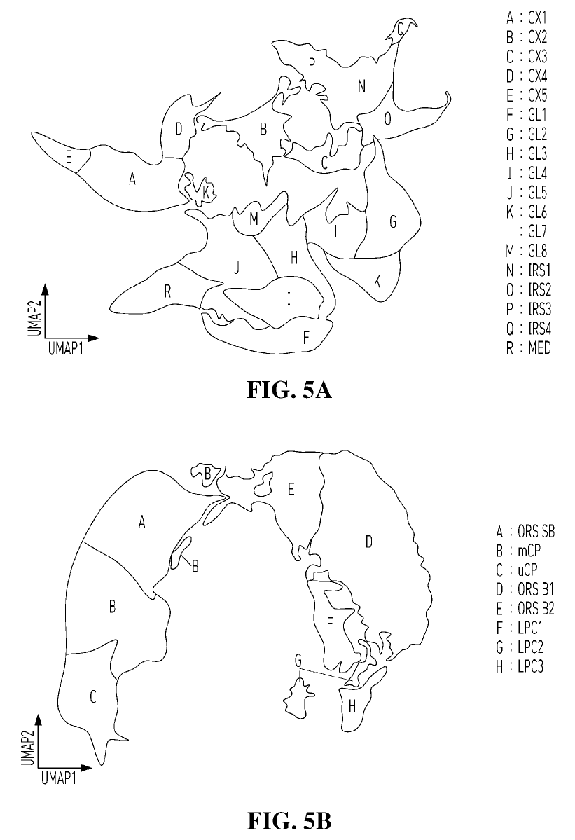

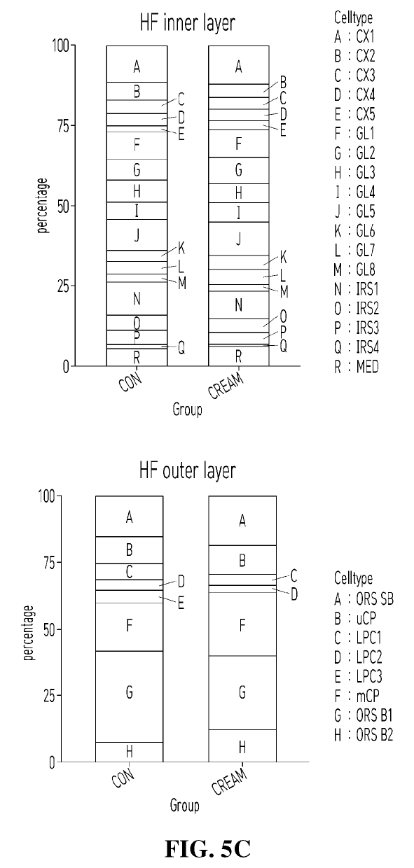

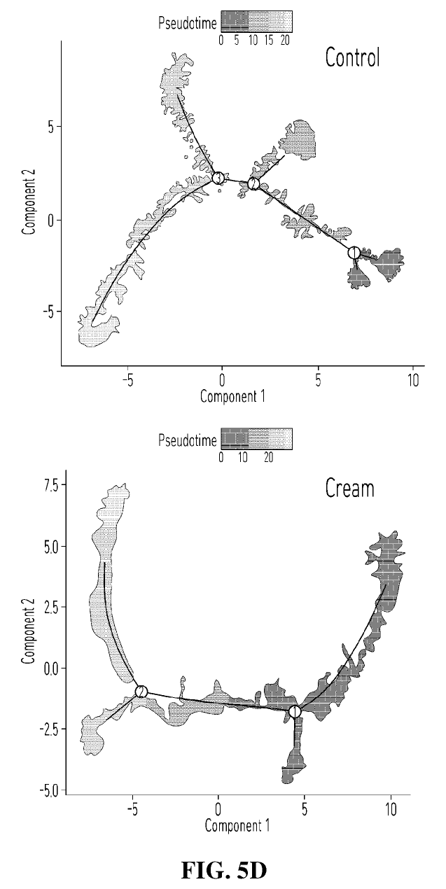

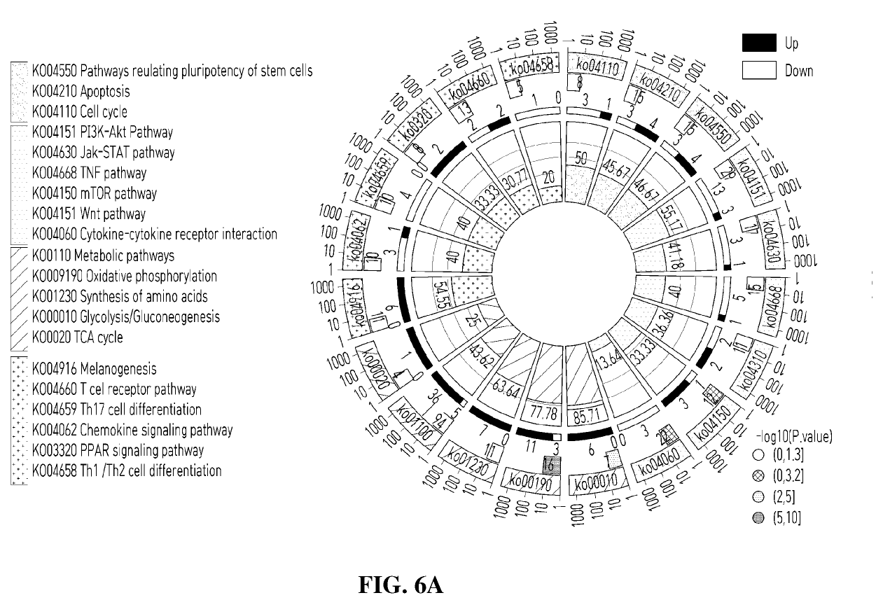

As a result, the inner layer of hair follicles consisted of clusters such as CX1–CX5, GL1–GL8, IRS1–IRS4, and MED (FIG. 5A), whereas the outer layer consisted of major subpopulations such as ORS SB, mCP, uCP, ORS B1, ORS B2, and LPC1–LPC3 (FIG. 5B). After CSMi treatment, changes in cell proportions within the inner layer were not particularly pronounced, with only a slight increase observed in the germinative layer (FIG. 5C). In contrast, the hair follicle outer layer exhibited marked changes, including a significant increase in the mid-part companion layer (mCP) and a significant decrease in the lower proximal cup (LPC) (FIG. 5C). The decrease in LPC concomitant with the increase in mCP suggests that these cells may differentiate into other cell types required for hair shaft formation. In addition, pseudotime trajectory analysis showed that CSMi cream treatment suppressed an initial minor differentiation node originating from mCP (FIG. 5D).

These findings suggest that the CSMi cream contributes to structural stability during hair follicle regeneration, extends the anagen phase, and protects stem cell reservoirs, thereby promoting hair growth.

### Example 6. Analysis of Mechanisms of Hair Follicle Activation

### 6-1. KEGG (Kyoto Encyclopedia of Genes and Genomes) Enrichment Analysis

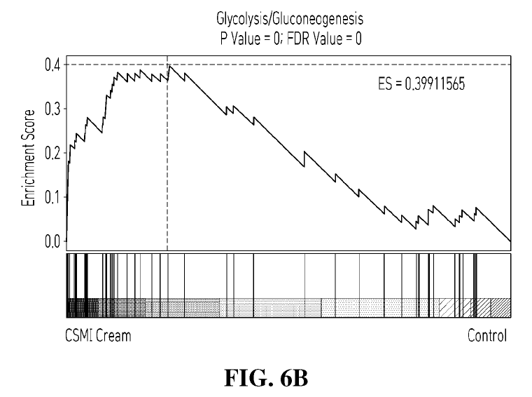

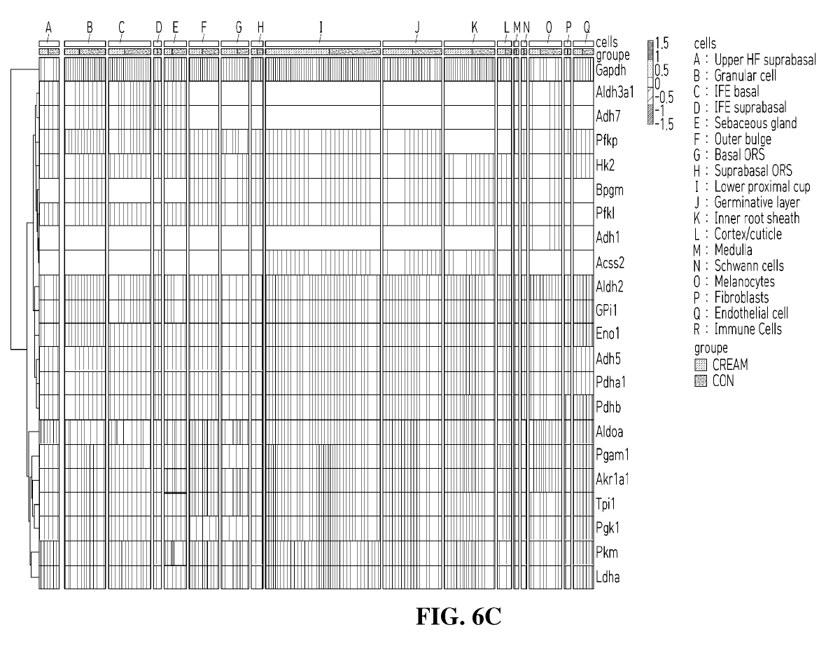

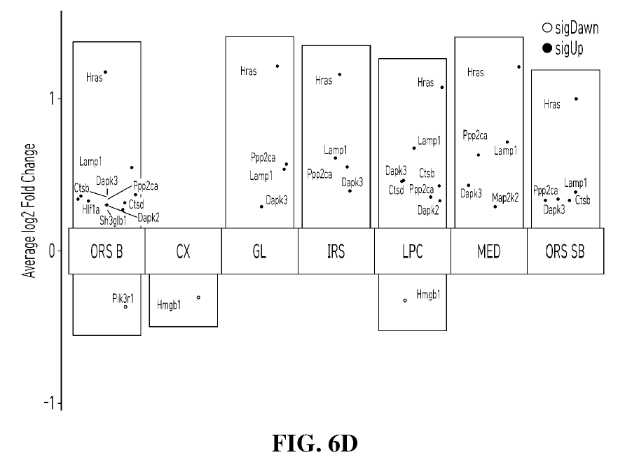

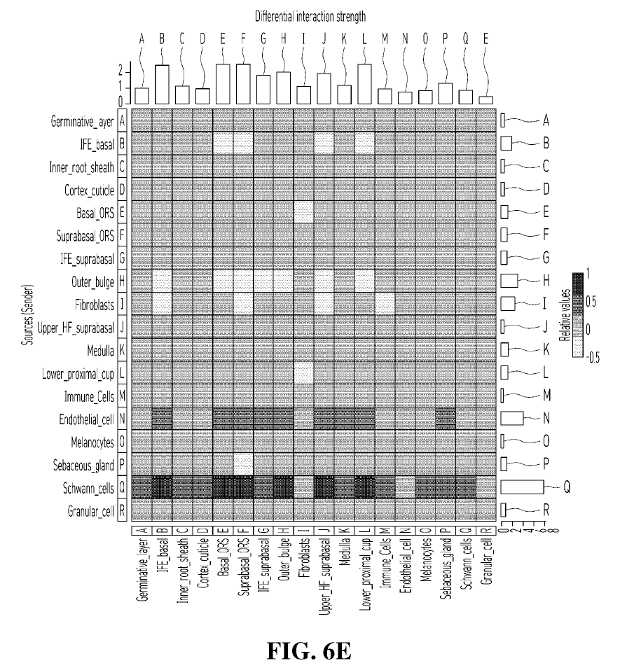

To examine changes in gene expression of specific pathways induced by treatment with the CSMi cream, KEGG enrichment analysis was performed. As a result, it was confirmed that the CSMi cream significantly affected gene expression in metabolic and related pathways (FIG. 6A). These effects were particularly prominent in pathways associated with metabolism and cellular processes, suggesting tendencies toward metabolic reprogramming and regulation of cellular functions.

The metabolic pathway category included a large number of genes, indicating that the CSMi cream can strongly activate multiple metabolic networks, including oxidative phosphorylation pathways, amino acid biosynthesis pathways, glycolysis/gluconeogenesis pathways, and apoptosis pathways. The significant changes observed in these metabolic and cellular process-related pathways may also be closely associated with interactions with other signaling pathways such as PI3K–Akt and mTOR.

### 6-2. GSEA (Gene Set Enrichment Analysis)

To further verify induction of glycolytic activity in skin and hair follicle-related tissues following treatment with the CSMi cream, GSEA (Gene Set Enrichment Analysis) was performed to evaluate increased expression of glycolysis-related genes in skin tissue. In addition, since autophagy, a core mechanism of intracellular degradation and recycling, is associated with glycolysis, expression of autophagy-related genes was also analyzed.

As a result, the glycolysis pathway was significantly enriched by CSMi cream treatment (FIG. 6B). In addition, major glycolytic enzyme genes were markedly upregulated in skin cells treated with the CSMi cream (FIG. 6C). Analysis of expression of autophagy-related genes revealed that the autophagy-related gene network was significantly activated by CSMi cream treatment (FIG. 6D). These genes exhibited tightly coordinated upregulation, potentially driving the entire autophagy process from initiation to degradation.

For example, upregulation of Ctsb, Ctsd, and Lamp1 collectively enhanced lysosomal function, suggesting more efficient degradation of autophagic contents. Increased expression of Sh3glb1 and Dapk3 suggested activation of the autophagy initiation stage, while simultaneous upregulation of Lamp1 and cathepsin family genes further strengthened overall autophagic flux from autophagosome formation to content degradation. Upregulation of Hif1a and HRas reflected metabolic remodeling under hypoxic or high-energy demand conditions, enhancing glycolysis and autophagy to provide rapid energy and metabolic support to cells. In addition, upregulation of Ppp2ca may further promote comprehensive autophagy activation by suppressing the mTOR signaling pathway. Collectively, these genes formed a highly coordinated signaling network that maximized cellular adaptability to external stimuli and metabolic changes.

### 6-3. Cell–Cell Interaction Analysis

In addition to glycolysis and autophagy within hair follicle stem cells (HFSCs), external cell–cell interactions may also influence cellular functions and processes. Accordingly, cell–cell interaction analysis was performed, and as a result, significant differences in probabilities of receptor–ligand pairs were observed between the untreated group and the CSMi cream-treated group (FIG. 6E).

In particular, interaction strength of Schwann cells as signal senders was enhanced in the CSMi cream-treated group, suggesting that the CSMi cream may directly or indirectly activate Schwann cells in the skin, thereby enhancing their activation state and increasing production and secretion of signaling molecules. In addition, analysis revealed that interactions between Schwann cells and basal ORS and LPC were most increased. Such enhanced communication may positively influence the hair growth cycle by extending the anagen phase and promoting transition from the telogen phase to the anagen phase.

### EXAMPLE 7. ANALYSIS OF MECHANISMS FOR EXTENSION OF THE ANAGEN PHASE

Since it was confirmed that the CSMi cream accelerates hair growth and promotes transition from the telogen phase to the anagen phase by activating hair follicle stem cells (HFSCs) through enhancement of autophagy and glycolysis, effects of the CSMi cream on the hair growth cycle were examined by DEG analysis and UMAP embedding analysis.

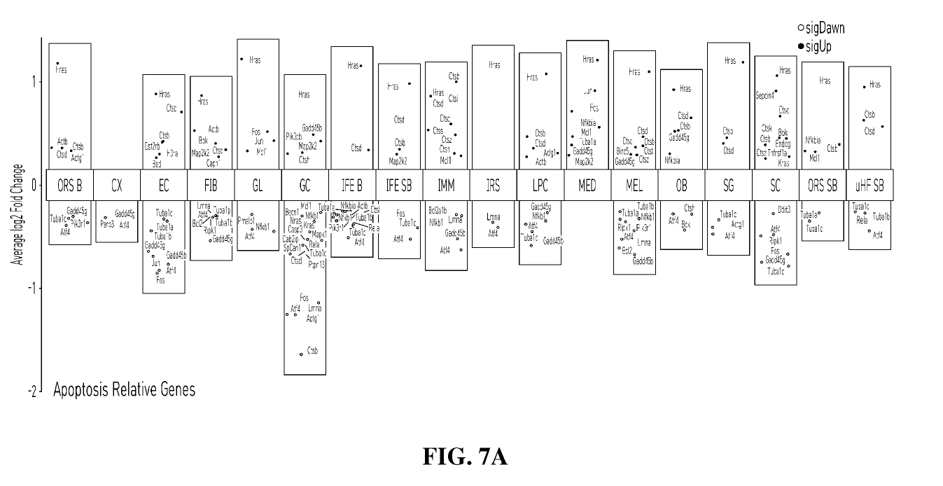

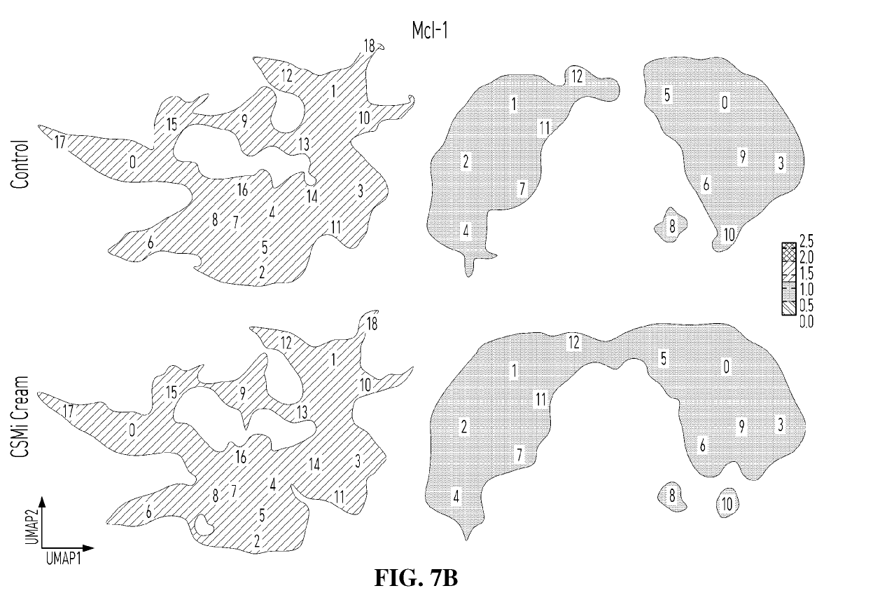

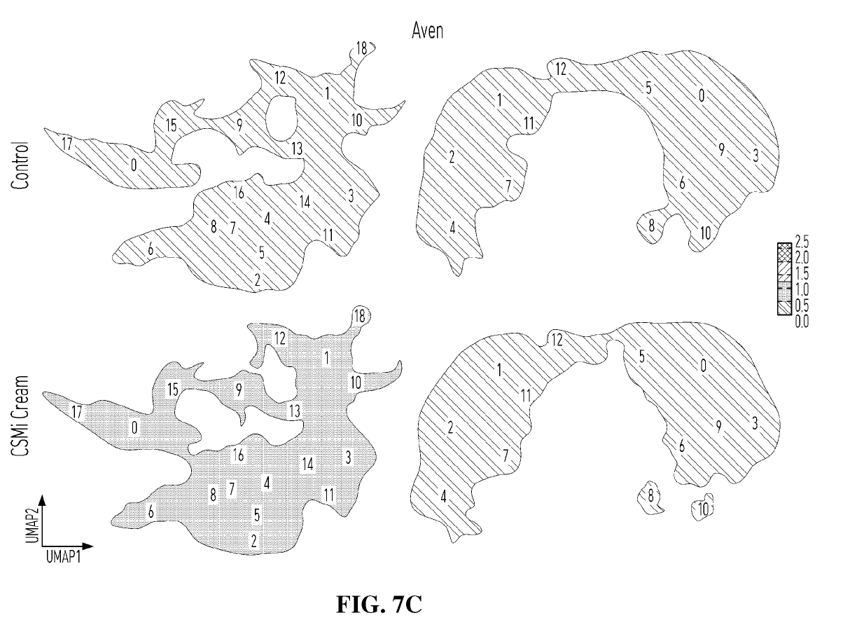

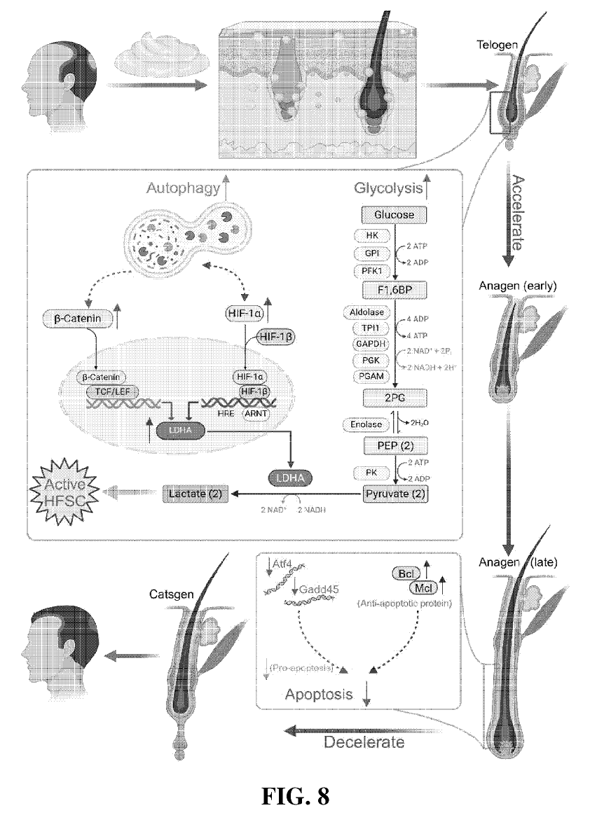

As a result, ATF4 was significantly downregulated in all detectable skin cells, including all cells within hair follicle structures, by treatment with the CSMi cream, and the Gadd45 family was also markedly decreased (FIG. 7A), suggesting inhibition of apoptosis. In addition, anti-apoptotic genes within hair follicles were specifically upregulated. For example, MCL1, a key anti-apoptotic protein of the BCL family, was significantly upregulated in both inner and outer layers of hair follicles (FIGS. 7A and 7B).

Furthermore, AVEN, which enhances anti-apoptotic functions by interacting with BCL family proteins and suppresses Apaf-1-mediated apoptosis, was specifically upregulated in GL, IRS, and CX regions (FIG. 7C), indicating that it may serve as a protective mechanism supporting survival and function of cells essential for hair growth within hair follicle regions. In addition, PARP1, an apoptosis-related gene, was significantly downregulated in the cortex/cuticle region (FIG. 7A).

These results indicate that the CSMi cream does not indiscriminately amplify glycolytic activity in hair follicles, but instead regulates interactions among glycolysis, autophagy, and apoptosis, thereby contributing to extension of the anagen phase and sustained promotion of hair growth.

What is claimed is:

1. A method for preventing or treating hair loss, comprising administering an effective amount of a pharmaceutical composition comprising chia seed mucilage to a subject in need of prevention or treatment of hair loss.

2. The method according to claim 1, wherein the chia seed mucilage is prepared by a method comprising:
(a) mixing chia seeds with distilled water and stirring;
(b) collecting mucilage; and
(c) filtering.

3. The method according to claim 1, wherein the composition comprises a gel comprising chia seed mucilage.

4. The method according to claim 3, wherein the gel is a chia seed mucilage polysaccharide (CSMP) gel prepared by a method comprising:
(a) mixing and stirring chia seed mucilage with PVA (poly(vinyl alcohol));
(b) adding and stirring D-sorbitol and glycerin; and
(c) performing UV treatment.

5. The method according to claim 1, wherein the composition further comprises chia seed oil (chia seed oil, CSO).

6. The method according to claim 5, wherein the composition comprises a gel comprising chia seed mucilage and a microencapsulation cream comprising chia seed oil.

7. The method according to claim 6, wherein the microencapsulation cream is a chia seed-based microencapsulation (CSMi) cream prepared by a method comprising:
(a) mixing and stirring SDS (sodium dodecyl sulfate) with chia seed oil;
(b) adding and stirring a gel comprising chia seed mucilage; and
(c) performing UV treatment.

8. The method according to claim 6, wherein microcapsules in the microencapsulation cream have a particle size of 500 to 5000 nm.

9. The method according to claim 1, wherein the administration induces promotion of hair follicle neogenesis, promotion of hair growth, promotion of transition of hair from a telogen phase to an anagen phase, or maintenance of an anagen phase.

10. The method according to claim 1, wherein the administration induces an increase in germinative layer cells, inner root sheath cells, outer root sheath cells, inner layer cells of hair follicles, medulla cells, or cortex/cuticle cells in the subject.

11. The method according to claim 1, wherein the administration increases expression of one or more selected from the group consisting of β-catenin, c-myc, ERK1/2, AVEN, Ctsb, Ctsd, Lamp1, Sh3glb1, Dapk3, cathepsin, Hif1a, HRas, Ppp2ca, MCL1, GLUT1, HK2, and PKM2 in the subject.

12. The method according to claim 1, wherein the administration decreases expression of one or more selected from the group consisting of ATF4, Gadd45, and PARP1 in the subject.

13. A pharmaceutical composition for preventing or treating hair loss, comprising chia seed mucilage.

14. The pharmaceutical composition according to claim 13, wherein the pharmaceutical composition comprises a gel comprising chia seed mucilage.

15. The pharmaceutical composition according to claim 13, wherein the pharmaceutical composition further comprises chia seed oil (chia seed oil, CSO).

16. The pharmaceutical composition according to claim 15, wherein the pharmaceutical composition comprises a gel comprising chia seed mucilage and a microencapsulation cream comprising chia seed oil.

17. A cosmetic composition for preventing hair loss or promoting hair growth, comprising chia seed mucilage.

18. The cosmetic composition according to claim 17, wherein the cosmetic composition comprises a gel comprising chia seed mucilage.

19. The cosmetic composition according to claim 17, wherein the cosmetic composition comprises a gel comprising chia seed mucilage and a microencapsulation cream comprising chia seed oil.

20. The cosmetic composition according to claim 17, wherein the cosmetic composition is formulated as a skin lotion, gel, aqueous liquid, cream, essence, oil-in-water (O/W) type formulation, or water-in-oil (W/O) type formulation.
